(19) United States
(12) Patent Application Publication
(10) Pub. No.: US 2007/0208102 A1
(43) Pub. Date: Sep. 6, 2007

(54) ANTIMICROBIAL PROSTHETIC DENTAL ELEMENT

(75) Inventors: Pierre-Luc Reynaud, Vaulnaveys Le Haut (FR); Manh-Quynh Chu, Fontanil Cornillon (FR); Cyril Rajon, Eybens (FR)

Correspondence Address: HESLIN ROTHENBERG FARLEY & MESITI PC, 5 COLUMBIA CIRCLE, ALBANY, NY, 12203, US

(73) Assignee: Recherches Et Techniques Dentaires - R.T.D., Saint Egreve (FR)

(21) Appl. No.: 11/680,822

(22) Filed: Mar. 1, 2007

(30) Foreign Application Priority Data
Mar. 2, 2006 (FR) 0650739

(51) Int. Cl. A61K 33/34; A61K 33/38; A61K 33/32; A61K 6/083

(52) U.S. Cl. 523/115; 424/618; 424/638; 424/641; 977/906

(57) ABSTRACT

Prosthetic dental element is obtained by machining a rod of composite material including fibres embedded in a matrix containing resin. The matrix also contains at least one agent with an antimicrobial effect at a concentration between 0.05 and 30% by weight of the prosthetic element. The agent is in powder form.

This invention concerns a prosthetic dental element made of composite material, said element having antimicrobial properties. The invention also concerns the production method for said prosthetic dental element.

“Prosthetic dental element” notably, but not restrictively, designates posts, bridge reinforcements and implant abutments.

In the rest of the description, the invention is more particularly described in relation to a dental post made of a composite material.

Dental posts are used for the reconstitution of pulpless teeth. A distinction is made between two types of posts, respectively:
metal or ceramic posts, and
composite posts.

Metal posts are usually made of stainless steel. Their main disadvantage is that they are subject to corrosion phenomena. Moreover, they have a transversal modulus of elasticity that is different from that of the dentine, leading to post separation over time.

To solve these problems, posts made of composite materials have been proposed, notably such as those described in the Applicant's document EP-A-432 001. In practice, these posts are constituted of long, unidirectional fibres made of glass or carbon and embedded in a thermosetting resin matrix. In general, the proportion of long fibres, whatever the material selected, accounts for 55 to 70% of the volume of the peg, the complement to 100% being occupied by the matrix.

To maintain the peg in the root, it must be glued and then sealed into said root. The practitioner then fills in the remaining spaces with a composite paste. The prosthesis or crown then covers the whole and is held in place by adding additional cement, if necessary accompanied by an adhesive.

Despite the care given to the various clinical steps in reconstituting the tooth, we can sometimes observe a recurrence of caries leading to, at best, a loosening of the prosthesis requiring complete retreatment of the root or, at worst, to the extraction of the tooth.

These recurring caries are due to an infiltration of pathogenic germs, such as Streptococcus mutans, Enterococcus faecalis, or even, in some cases, Staphylococcus aureus. Such infiltration may be due to loss of marginal seal between the prosthesis, the dentine and the filling material. Sources of bacteria may also subsist when dentine etching is poorly executed and lets debris appear. Germs can also take advantage of natural obstacles such as undercuts, working as receptacles.

To solve this problem, several solutions have been developed, such as antimicrobial adhesives or cements, but also etching solutions.

Thus, document U.S. Pat. No. 5,385,728 describes an acid etching composition containing benzalkonium chloride as an antimicrobial agent.

Document U.S. Pat. No. 5,968,253 discloses a calcium phosphate-based dental cement which may contain an antimicrobial agent in the form of an antibiotic or an antiamebic.

Document U.S. Pat. No. 6,326,417 concerns an antimicrobial dental composition used as a restorative adhesive, cement or composite paste containing compounds of salicylic acid or sulfanilamide, possibly in derived form.

Documents U.S. Pat. No. 5,408,022, U.S. Pat. No. 5,494,987 and U.S. Pat. No. 5,733,949 describe polymer-based compositions with antimicrobial properties after polymerisation, used to produce artificial skin, catheters, contact lenses as well as dental products, such as adhesives, resins and composites. The antimicrobial agent is a polymerisable monomer, whose active group is based on a quaternary salt (chlorine or bromine).

Document U.S. Pat. No. 6,355,704 describes an antimicrobial dental adhesive with two components. One of the components is an antimicrobial polymerisable monomer with a cationic group selected from the ammonium, pyridinium and phosphonium bases.

Document U.S. Pat. No. 6,924,325 describes an antimicrobial dental composition which can be used as a restorative endodontic, orthodontic or prosthetic composition. This composition contains as its active ingredient a ceramic glass/zinc-silver powder or a silver-based zeolite powder.

Document U.S. Pat. No. 6,071,528 describes an antibacterial dental cement adhesive whose active ingredient is selected among antibiotics, alkali metal hydroxides, alkaline earth metal hydroxides or alkaline earth metal oxides.

Products of other types than the aforementioned with antimicrobial properties have been developed. These are notably compositions used for whitening teeth, as described in documents U.S. Pat. No. 5,985,249, U.S. Pat. No. 6,036,943, U.S. Pat. No. 6,309,625 and U.S. Pat. No. 6,368,576, in which the antimicrobial agent is selected from among chlorhexidine, benzoate derivatives, tetracycline or fluorinated compounds.

Document U.S. Pat. No. 6,365,130 describes a chewing gum containing antimicrobial ceramic particles based on metal cations.

Document U.S. Pat. No. 6,267,590 deals with a bracket and an orthodontic wire treated by the deposition of a silver zeolite ceramic polymer.

Document U.S. Pat. No. 6,113,993 deals with a method for treating a dental implant, usually metal, said treatment consisting in applying to the surface of the implant, a calcium phosphate-type binding agent. Once the treatment has been applied, it is proposed that the implants should be soaked in an antimicrobial agent solution before implanting.

Document U.S. Pat. No. 6,113,993 concerns a method for treating a dental implant, usually metal, said treatment consisting in applying a calcium phosphate binding agent to the surface of the implant. Once treated, it is proposed that the implants should be soaked in a solution of an antimicrobial agent before implantation.

Documents WO 02/100355 and WO 03/105785 describe prepregs treated with an antimicrobial agent. The prepregs are “pre-impregnated” comprised of reinforcement fibres impregnated in a resin matrix. The prepregs are supplied to the practitioner or prosthetist as such, who themselves perform the curing of the material when inserting it into the mouth. These documents mention the possibility of using these prepregs as pegs. In this hypothesis, they will not be machined to obtain a precise, predefined shape as meant by prosthetic element in this invention, as it is the practitioner himself who manually gives it its final shape.

In document WO 02/100355, an antimicrobial agent is applied to the surface of the prepreg. In document WO 03/105785, the antimicrobial agent is applied as a liquid in the pores of a resin matrix produced according to the disclosure of document U.S. Pat. No. 5,846,640. In practice, a “porous” matrix is prepared, the antimicrobial liquid is incorporated into the pores, then the remaining pore volume is “plugged” with a curable material. The method thus requires many steps.

Document WO 96/15759 describes tooth posts containing metal oxides in the matrix which can provide the post with a certain degree of radiopacity to X-rays, the metal oxide content being expressed in relation to the weight of the matrix. No indication is given as to any antimicrobial properties of these oxides.

The Applicant has observed that it is possible to cause a prosthetic element to become antimicrobial by adding to its matrix at least one antimicrobial agent in a particular form at 0.05 to 30% by weight of the element, keeping its effect on the mechanical properties to a minimum.

In other words, the subject of the invention is a prosthetic dental element obtained by machining a rod of composite material including fibres embedded in a matrix containing resin. This prosthetic element is characterised in that the matrix also contains at least one agent with an antimicrobial effect at a concentration between 0.05 and 30% by weight of the prosthetic element, said agent being in powder form.

Above 30%, the mechanical properties are unsatisfactory and the antimicrobial effect reaches a plateau. Of course, the lower the concentration, the better the mechanical characteristics. Below 0.05% by weight, the antimicrobial effect is not observed.

In a particular embodiment, the prosthetic element is a peg. In practice, the post comprises reinforcement fibres embedded in a matrix containing resin. These may be glass, carbon, ceramic or quartz fibres.

In practice, the antimicrobial agent is selected from the group including zinc oxide, copper oxide and silver oxide.

To obtain a more homogenous distribution in the matrix and not have a localised constraint by being more voluminous than the diameter of the filaments of the reinforcement fibres, the antimicrobial agent comes in the form of extremely fine particles, preferably with a median diameter D50 (median diameter of 50% of the particles) with a value of less than 10 μm, advantageously less than 5 μm, preferably approximately 1 μm. In another embodiment, the particles have a nanometric size between 30 and 80 nm.

Advantageously, the antimicrobial agent accounts for 2 to 10% by weight of the prosthetic element.

At the same time, the matrix generally accounts for 30 to 40% by volume of the prosthetic element or 20 to 45% by weight.

In one advantageous embodiment, the prosthetic element is not only antimicrobial but also radiopaque.

To make the product visible to X rays, the prosthetic element contains at least one radiopaque substance.

Those skilled in the art know of several ways of making the product radiopaque.

In a first embodiment, at least one radiopaque substance is incorporated into the fibres. In this case, the fibres used may have a calcium oxide or zirconium oxide base.

In a second embodiment, the radiopaque substance is incorporated into the matrix. In some cases, the antimicrobial agent and the radiopaque substance are the same product.

This particular embodiment provides the advantage of preserving mechanical properties and simplifying production of the rod. In general, the decrease in mechanical characteristics is proportional to the quantity of filler added to the matrix. Thanks to this twofold function, it is useful to combine several antimicrobial agents so as to obtain a broad-spectrum antimicrobial material, i.e. active against strains of Streptococcus mutans and Enterococcus faecalis, while preserving mechanical properties and radiopacity.

In a third embodiment, the radiopaque substance is added to the resin and to the fibres. In practice, according to the invention, the radiopaque substance is selected from the group including metal oxides such as aluminium oxide, barium oxide, strontium oxide, tungsten oxide, zinc oxide, zirconium oxide or fluorinated compounds, such as ytterbium and yttrium or carbonates such as lanthanum carbonate and zirconium carbonate, alone or in mixtures.

The antimicrobial agent is added to the resin matrix, which will then impregnate and link all of the fibres together. The composite material thus obtained will have at least one antibacterial agent embedded in its mass. After machining, the rod may be a dental post, a bridge reinforcement or an implant abutment or any other prefabricated dental element.

When the devices are posts or bridge reinforcements, the practitioner is often required to cut and mill them at least partially to adapt them to the anatomy. As the matrix contains an antibacterial agent, the surface exposed still has antibacterial activity.

The resin matrixes are polymers with an epoxy, methacrylate, polyester or vinylester resin base.

The reinforcement fibres may be carbon, glass, quartz or silica fibres.

The rod is produced using the technique called pultrusion, well known to those skilled in the art. Its cross section varies depending on the finished product to be machined.

To obtain the final shapes of the posts, bridge reinforcements, etc., machining techniques well known to those skilled in the art, such as turning or numerical control, are used.

The invention and the resulting advantages will be made clear by the following non-restrictive examples of embodiments.

### EXAMPLE 1

The rod with the following composition is produced:
zinc oxide, 99% pure, 1 μm size (mean D50): 5% by weight
Bisphenol A-based epoxy resin matrix: 29% by weight
E-glass fibres: 66% by weight

Three coated 8-mm diameter samples taken from this rod underwent antimicrobial activity tests at the same time as three untreated samples used as controls. The test method is inspired by Japanese Industrial Standard JIS Z 2801 “Antimicrobial product-Test for antimicrobial activity and efficacy”.

The bacterial strain used was Staphylococcus aureus.

All samples were incubated for 24 h at 38° C. The populations of Staphylococcus aureus were then counted. Antimicrobial activity is equal to the log of the difference between the number of bacteria counted in the control sample minus the number of bacteria counted in the sample tested. This antibacterial activity is expressed in log power.

The samples from this example phagocyted practically all of the bacteria, as 2.19 log germs were counted after 24 h of incubation whereas the number is 6.58 log for the control samples. The difference between these two averages corresponds to the bactericidal activity of the sample tested.

Bactericidal activity is 4.39 log on Staphylococcus aureus.

The rod is then machined to obtain dental posts.

### EXAMPLE 2

The rod with the following composition is produced:
zinc oxide, 99% pure, 1 μm size (mean D50): 8% by weight
glass fillers containing strontium oxide, zirconium oxide, 1 μm size (mean D90): 4%
Bisphenol A-based epoxy resin matrix: 30% by weight
E-glass fibres: 58% by weight

The samples from this example undergo the antibacterial activity tests described in the previous example.

Bactericidal activity is 3.49 log on Staphylococcus aureus.

### EXAMPLE 3

In this example, the antimicrobial character of the rods containing certain metal oxides is tested against a strain of Streptococcus mutans. The tests are performed according to standard J15Z2801 “Antimicrobial products. Test for antimicrobial activity and efficacity”.

The rods have the following composition:
metal oxide: 5% by weight
Bisphenol A-based epoxy resin matrix: 37% by weight
E-glass fibres: 58% by weight

| | | Activity |
|---|---|---|
| Active ingredient | Results | (Log) |
| Magnesium oxide | Inactive | 0.42 |
| Copper oxide | Bactericidal | 3.94 |
| Silver oxide | Bactericidal | 2.67 |

These results show that, for a very weak concentration of approximately 5% by weight, the antimicrobial effect is obtained with copper oxide and silver oxide. At this concentration, the decrease in the mechanical characteristics of the post remains acceptable.

1/ Prosthetic dental element comprising a machined rod of composite material including fibres embedded in a matrix containing resin, wherein the matrix also contains at least one agent with an antimicrobial effect at a concentration between 0.05 and 30% by weight of the prosthetic element, said agent being in powder form.

2/ Prosthetic element as claimed in claim 1, wherein the at least one agent with an antimicrobial effect accounts for 2 to 10% by weight of the prosthetic element.

3/ Prosthetic element as claimed in claim 2, wherein the at least one agent with an antimicrobial effect is selected from the group consisting of zinc oxide, copper oxide and silver oxide.

4/ Prosthetic element as claimed in claim 1, wherein a median diameter D50 of the the at least one agent with an antimicrobial effect has a value of less than 10 μm.

5/ Prosthetic element as claimed in claim 1, wherein particles of the at least one agent with an antimicrobial effect have a nanometric size between 30 and 80 nm.

6/ Prosthetic element as claimed in claim 1, wherein the matrix accounts for 20 to 45% by weight of the prosthetic element.

7/ Prosthetic element as claimed in claim 1, wherein the prosthetic element comprises a post, a bridge reinforcement or an implant abutment.

8/ Prosthetic element as claimed in claim 4, wherein the median diameter D50 of the at least one agent with an antimicrobial effect has a value of less than 5 μm.

9/ Prosthetic element as claimed in claim 4, wherein the median diameter D50 of the at least one agent with an antimicrobial effect has a value of approximately 1 μm.
